(19) United States
(12) Patent Application Publication
(10) Pub. No.: US 2013/0157275 A1
(43) Pub. Date: Jun. 20, 2013

(54) OLIGONUCLEOTIDE MARKER AND METHOD FOR IDENTIFYING THE SAME

(71) Applicant: BIONEER CORPORATION, Daejeon (KR)

(72) Inventors: Han Oh PARK, Daejeon (KR); Jae-Don LEE, Seongnam-si (KR); Gu-Young SONG, Daejeon (KR); Jong Wak OH, Cheongju-si (KR); Hyun-Bae KIM, Daejeon (KR); Sang-Jin BYUN, Daejeon (KR); Jin-Wong JANG, Cheongju-si (KR); Won-Seok JANG, Daejeon (KR); Jong-Deok CHOI, Daejeon (KR)

(73) Assignee: BIONEER CORPORATION, Daejeon (KR)

(21) Appl. No.: 13/782,796

(22) Filed: Mar. 1, 2013

Related U.S. Application Data
(63) Continuation of application No. PCT/KR2011/006540, filed on Sep. 2, 2011.

(30) Foreign Application Priority Data
Sep. 3, 2010 (KR) 10-2010-0086468

(51) Int. Cl. C12Q 1/68; C07H 21/04

(52) U.S. Cl. 435/6.12; 536/23.1

(57) ABSTRACT

Provided are an oligonucleotide marker and a method of identifying a material using the same. The oligonucleotide marker makes it possible to analyze a trace amount of the material with high precision within a short time, has improved solubility in an oily solvent, and can improve a detection method such that the oligonucleotide marker can be detected within 2 hours. The oligonucleotide marker can label various products, including oil products and petroleum products, works of art and collections, and can also be used to conduct criminal investigations.

### TECHNICAL FIELD

The present invention relates to an oligonucleotide marker and a method for identifying the same.

### BACKGROUND ART

Oligonucleotides have unique advantages because they can be amplified in large amounts even when they are present in very small amounts by polymerase chain reaction (PCR) and because the original nucleotide sequences thereof can be determined by nucleotide sequencing. Thus, when such oligonucleotides are added in very small amounts to various materials or products, including oils, paints, explosives, and works of art, the original source or transport pathway of the materials or products or whether the products are authentic can be precisely determined.

To label oil products, methods of adding a fluorescent reagent, pigments or specific chemical substances are generally used. However, there are problems in that quantitative analysis for very small amounts of samples is difficult, the labeling of various products is difficult, contamination by operators can occur, and manipulation such as the removal of the labels is possible.

A standard synthesis method which is used in an oligonucleotide synthesizer is the phosphitetriester method. In the phosphitetriester method, a phosphodiester bond that forms the backbone of the DNA structure is made using β-cyanoethyl phosphoramidite. In this method, an oligonucleotide having the desired length is synthesized by repeating a synthesis process consisting of deblocking, coupling, capping and oxidation, beginning from a solid support having nucleoside attached thereto. The deblocking step which is the first step of the synthesis process starts with detaching DMT from the solid support, and the 5′-hydroxyl group produced in the deblocking step undergoes a coupling reaction with a nucleoside phosphoramidite monomer to synthesize an oligonucleotide having the desired nucleotide sequence. The deblocking step is carried out under acidic conditions using trichloroacetic acid or dichloroacetic acid. After the coupling step, the unreacted 5′-hydroxyl group can participate in the next coupling step to produce (n−1)mer having an undesired nucleotide sequence, and for this reason, the unreacted 5′-hydroxyl group is capped by acetylation with acetic anhydride and N-methylimidazole. The structure resulting from the coupling step is a phosphite ester and is oxidized with iodine so that it is converted into a phosphate ester form which is a part of the structure of actual DNA. Repeating the above synthesis process allows an oligonucleotide having a desired length to be synthesized. After completion of the synthesis, the synthesized oligonucleotide is detached from the solid support by treatment with ammonia, and the β-cyanoethoxy group is removed therefrom so that the synthesized oligonucleotide is restored to a phosphodiester bond forming the backbone of the DNA structure.

Because a phosphodiester bond is negatively charged at neutral pH, an oligonucleotide consisting of a plurality of phosphodiester bonds shows strong hydrophilic properties.

Thus, the oligonucleotide easily dissolves in an aqueous solution, but is generally insoluble in organic solvents. This property causes the problem of poor solubility when the oligonucleotide is dissolved in an organic solvent.

With respect to methods of using such DNA to label objects, WO87/06383 discloses that nucleotides can be used as labels, but a method of identifying an object by DNA amplification or sequencing is not disclosed and a method of dissolving hydrophilic DNA in an organic solvent is not disclosed. WO90/14441 discloses a technique of introducing hydrophilic DNA into an organic layer using a detergent to dissolve the DNA in oil. However, WO90/14441 merely discloses that the presence or absence of DNA is determined by using specific primers to examine whether the DNA was amplified. Also, it fails to mention using the nucleotide sequence of DNA as an identification marker. Moreover, when the detergent is used, DNA is present in the form of a reverse micelle in the organic layer so that it agglomerates without being dispersed at the molecular level. In addition, the DNA introduced into the organic layer in the reverse micelle form can be easily extracted into a water layer so that it is likely to be removed.

WO91/17265 discloses determining the nucleotide sequence of a gene by amplifying the gene with the specific primers described in WO90/14441, and also discloses that the DNA can be covalently bonded with a solid support or material. With respect to the disclosure of WO91/17265, when nucleotides are bonded directly to paints or oils, the covalent bonds should be broken in the process of extracting and collecting the oligonucleotide, thereby modifying the nucleotides. For this reason, the oligonucleotide is not amplified into an exact sequence, and thus they are difficult to commercialize.

WO94/14918 discloses a more improved method of amplifying and sequencing a gene and using, as labels, two or more light-emitting materials or compounds emitting colors. However, this method also does not consider the reactivity of the hydroxyl group of an oligonucleotide or the amino group of the nucleotide. Due to the reaction of the hydroxyl group or amino group moiety, an oligonucleotide having the original sequence cannot be obtained when polymerase chain reaction (PCR) or nucleotide sequencing is performed.

U.S. Pat. No. 5,665,538 discloses a method of monitoring the movement of a petroleum material in an aqueous solution, comprising adding a microtrace additive to the petroleum material. The microtrace additive is added to the petroleum material at a final concentration of 0.01-1000 pg/DNA/ul using DNA. The DNA is formulated to be soluble in the petroleum material such that the hydrophobicity of the microtrace additive causes it to partition into the petroleum material. The formulation ensures that the DNA is dissolved in or dispersed within the petroleum material such that it essentially cannot be removed by aqueous washing. The microtrace additive-containing petroleum material is sampled after it moves, and then the microtrace additive is removed from the petroleum material, and finally the DNA microtrace additive is detected by means of an amplification reaction.

US Patent Publication 2007/0065876 discloses a marking system comprising a combination of oligonucleotides having different sizes. Each of the DNAs comprises three fragments, in which the middle fragments have different lengths varying depending on the lengths of the oligonucleotides so that such different lengths serve as codes, and both end fragments are primers having different sequences. The primers serve as detection elements to determine the presence or absence of a material. The oligonucleotide DNA is detected by amplification.

U.S. Pat. No. 5,451,505 discloses a method of monitoring the presence of a substance exposed to naturally occurring ultraviolet radiation which comprises tagging the substance, such as an air pollutant, oil or aromatic compound, with a nucleic acid of at least 20 and less than 1,000 nucleotides, releasing the tagged substance in such a manner that said substance and nucleic acid are exposed to naturally occurring ultraviolet radiation, collecting the nucleic acid, amplifying said nucleic acid using the polymerase chain reaction, and monitoring the presence of the substance.

EP1171633 discloses a nucleotide tag comprising the same probe sequences and different primer sequences and also discloses a nucleotide tag sequence in which the forward primer and probe are fixed while the reverse primer is varied. Herein, the nucleotide tag in the sample is quantitatively detected by PCR using primers and fluorescence-labeled tags, thereby allowing the amount of the marker in the material to be quantitatively determining.

In an attempt to overcome the above-described limitations of the prior art, Korean Patent Registration No. 10-0851764 registered in the name of the applicant discloses an oligonucleotide having improved solubility in a lipophilic solvent and a method of identifying a material using the same. Also, Korean Patent Registration No. 10-0851765 registered in the name of the applicant discloses an oligonucleotide marker, which is added to a vehicle paint film and which is suitable for use as a vehicle identification marker, and a method of detecting a vehicle using the same. According to the disclosed method, a material can be tracked and monitored using nucleotide sequence information by extracting a trace amount of an oligonucleotide dissolved in paint, collecting the oligonucleotide, amplifying the collected oligonucleotide by PCR, and sequencing the amplified oligonucleotide. In this method, a process of decoding the nucleotide sequence is required, because the sequence information is encoded. The method of analyzing the nucleotide sequence is not easy to commercialize because of the analysis cost, precision, time consumption, and complex processes, and there are limitations to determining whether a material is authentic and to determining the coding information of a material (an internally-used identification number such as lot no., manufacturer, etc.) in an easy and rapid manner.

In addition, as oils having various qualities are marketed, the manipulation of oil grades and the circulation of non-standard gasoline are problematic. Thus, to manage the brand image of a manufacturer and apply order to the circulation of these, a technique capable of identifying the kind and quality of oil being circulated is required.

### DISCLOSURE

### Technical Problem

An object of the present invention is to provide a more stable oligonucleotide and a method of labeling a material with the oligonucleotide and recovering and identifying the labeled oligonucleotide marker from the material within a short time.

### Technical Solution

In one aspect, the present invention provides an oligonucleotide linked with a cationic phase transfer agent which is a quaternary ammonium salt compound or a cationic surfactant, and a method of labeling a material with the oligonucleotide and recovering and identifying the labeled oligonucleotide marker from the material.

Hereinafter, the present invention will be described in detail. In one aspect, the present invention provides an oligonucleotide marker for identifying a material, the oligonucleotide marker being an oligonucleotide linked with a cationic phase transfer agent which is a quaternary ammonium salt compound or a cationic surfactant the oilgonucleotide marker comprising a probe sequence for real-time polymerase chain reaction (PCR) and primer sequences linked to both ends of the probe sequence.

In the present invention, the cationic phase transfer agent may be a quaternary alkylammonium ion, such as tetrabutylammonium hydroxide or hexadecyltrimethylammonium bromide.

In the present invention, the oligonucleotide may be blocked at its end by a blocking agent, in which the blocking agent is free of a chemical substance or end group, such as lipid or phosphate.

In the present invention, the oligonucleotide comprises reactive nitrogen and oxygen moieties linked to an organic compound containing 1-50 carbon atoms, in which the organic compound containing 1-50 carbon atoms is any one selected from the group consisting of a carbonyl compound forming an amide bond with nitrogen and an ester bond with oxygen, a silanyl compound forming an O—Si bond with N—Si, a sulfonyl compound forming an O—S bond with N—S, and a saturated hydrocarbon, aromatic hydrocarbon, unsaturated hydrocarbon, or heteroatom-containing saturated or unsaturated hydrocarbon compound which forms an O—C bond with N—C in which the bond between N—C and O—C may be broken by treatment with ammonia.

In the present invention, the oligonucleotide has a length of 20-1000 nucleotides.

In another aspect, the present invention provides a method for identifying a material, the method comprising adding said oligonucleotide marker to the material.

The material may be one selected from the group consisting of paints for vehicle coating, lacquers, paints for traffic lines, petroleum, paint diluents, thinners, explosives, naturally occurring oils, paints for construction, organic solvents, adhesives, dyes, meats and fishes.

In another aspect, the present invention provides a method for identifying a material, the method comprising:

1) extracting an oligonucleotide marker from a material labeled with the oligonucleotide marker, the oligonucleotide marker being an oligonucleotide linked with a cationic phase transfer agent which is a quaternary ammonium salt compound or a cationic surfactant, the oligonucleotide marker comprising a probe sequence for real-time polymerase chain reaction (PCR) and primer sequences linked to both ends of the probe sequence;

2) amplifying the extracted oligonucleotide by real-time PCR using the primers and the probe; and

3) using the real-time PCR product to identify the material labeled with the oligonucleotide marker.

In the present invention, the probe may be a fluorescence-labeled probe.

In the present invention, the extracting of 1) may be performed by adding an anionic surfactant to the material.

In the present invention, the material may be a fat-soluble or water-soluble material.

More specifically, the material may be one selected from the group consisting of paints for vehicle coating, lacquers, paints for traffic lines, petroleum, paint diluents, thinners, explosives, naturally occurring oils, paints for construction, organic solvents, adhesives, dyes, meats and fishes.

In the present invention, the identifying may be performed by identifying the material by a combination of the oligonucleotide markers having different sequences, which are contained in the material.

In still another aspect, the present invention provides a composition for identifying a material, the composition containing said oligonucleotide marker.

In the present invention, the composition may further comprise a primer and probes corresponding to the oligonucleotide marker.

### Advantageous Effects

The oligonucleotide marker for identifying a material according to the present invention makes it possible to analyze a trace amount of the material with high precision within a short time, has improved solubility in an oily solvent, and can improve the detection method such that the oligonucleotide marker can be detected within 2 hours.

The method for identifying the material according to the present invention can provide labeling that is several hundred times more sensitive than a conventional labeling method that uses sequencing or a conventional method of labeling with fluorescent dyes, while it can label various products, thus making it possible to perform management on a product basis in actual production processes.

The oligonucleotide marker for identifying the material according to the present invention can label various products, including oil products and petroleum products, works of art and collections, and can also be used to conduct criminal investigations.

### DESCRIPTION OF DRAWINGS

The above and other objects, features and further advantages of the present invention will become apparent from the following description of preferred embodiments given in conjunction with the accompanying drawings, in which:

FIG. 1 shows an oligonucleotide linked with a phase transfer agent, in which the nucleotide moiety or alcohol in 5 and 3 regions of the oligonucleotide forms amide and ester bonds (R: C₁˜C₁₈);

FIG. 2 shows the results of analyzing the solubility of an oligonucleotide in an organic solvent as a function of the use of a cationic phase transfer agent ((A): the amount of oligonucleotide dissolved in diesel; (B): the amount of oligonucleotide dissolved in gasoline; (C): the amount of lipid-containing oligonucleotide dissolved in diesel; and (D): the amount of lipid-containing oligonucleotide dissolved in gasoline);

FIG. 3 shows the results of analyzing the recovery of the oligonucleotide of the present invention dissolved in the organic solvent gasoline ((A): the recovery of oligonucleotide dissolved in diesel; and (B): the recovery of oligonucleotide dissolved in gasoline);

FIG. 4 FIG. 3 shows the results of analyzing the recovery of the oligonucleotide of the present invention dissolved in diesel as an organic solvent;

FIG. 5 shows the results of MALDI-TOF Mass analysis conducted to examine the modification of the molecular structure of the inventive oligonucleotide recovered from the organic solvent gasoline;

FIG. 6 shows the results of MALDI-TOF Mass analysis conducted to examine the modification of the molecular structure of the inventive oligonucleotide recovered from the organic solvent diesel;

FIG. 7 is a set of graphs showing a fluorescence curve (A) for a real-time quantitative nucleic acid amplification reaction with the oligonucleotide of the present invention, and the linearity (B) of a quantification curve plotted using the fluorescence curves;

FIG. 8 is a set of graphs showing fluorescence curves (A) for real-time quantitative nucleic acid amplification reactions conducted using serially diluted standard oligonucleotides as templates with probes and primers, as a function of copy number, and the linearity (B) of a quantification curve plotted using the fluorescence curves;

FIG. 9 is a set of graphs showing fluorescence curves (A) for real-time quantitative nucleic acid amplification reactions conducted using oligonucleotide markers and standard oligonucleotides, purified in gasoline, as templates with probes and primers, as a function of copy number, and the linearity (B) of a quantification curve plotted using the fluorescence curves;

FIG. 10 is a schematic diagram showing identification information for the oligonucleotide marker of the present invention;

FIG. 11 is a schematic view showing whether a sample labeled with four different primer sets and five probe sets produces various identification codes in Example 4 of the present invention (primer set 1: red, primer set 2: yellow, primer set 3: green, primer set 4: blue, probe 1: purple, probe 2: blue, probe 3: green, probe 4: orange, and probe 5: light green);

FIG. 12 is a graph showing the results of quantitative analysis conducted by means of a qPCR reaction using a probe set of SEQ ID NOS: 35 and 39, a primer set of SEQ ID NOS: 27 and 28 and a primer set of SEQ ID NOS: 29 and 30 as a control in Example 4 of the present invention;

FIG. 13 is a graph showing the results of quantitative analysis conducted by means of a qPCR reaction using a probe set of SEQ ID NOS: 35 and 39 and a primer set of SEQ ID NOS: 27 and 28 for four templates in Example 4 of the present invention; and

FIG. 13 is a graph showing the results of quantitative analysis conducted by means of a qPCR reaction using a probe set of SEQ ID NOS: 35 and 39 and a primer set of SEQ ID NOS: 29 and 30 for four templates in Example 4 of the present invention.

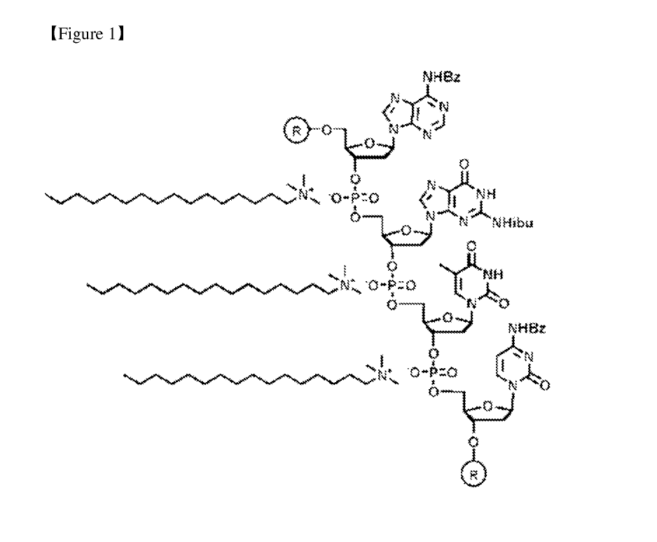

### BEST MODE

Hereinafter, the present invention will be described in further detail with reference to examples. However, these examples are for illustrative purposes only and the scope of the present invention is not limited to these examples in any way.

Unless defined otherwise, all technical and scientific terms used herein have the same meaning as commonly understood by one of ordinary skill in the art to which this invention belongs. In the following description and the accompanying drawings, the description of known functions and constructions that would make the subject matter of the present invention unnecessarily ambiguous will be omitted.

### Example 1

### Dissolution of Oligonucleotide in Organic Solvent

1) Preparation of Oligonucleotide

In order to examine the solubility of an oligonucleotide in an organic solvent by a phase transfer agent (PTA), an oligonucleotide having a desired nucleotide sequence was synthesized on controlled pore glass (CPG) using an automatic synthesis system. The oligonucleotide sequence was designed such that it could be analyzed using qPCR (quantitative polymerase chain reaction), and it was a template DNA having a length of 68 mer.

SEQ ID NO 1(normal-68 mer):
5′-ATTCGGTGAATAAGCACTCTCATAGTCCTCATCCAACTGCGCGTCTTGCATAGAG CTGCTGACCCTAC-3′ (MW=20777).

To improve the solubility of the oligonucleotide as a template in an organic solvent and to stabilize the oligonucleotide in oils, the oligonucleotide may be synthesized to have a lipid added to both ends thereof. In this Example, the oligonucleotide was designed such that it further comprises C12 at the 3′ end and C18 at the 5′ end.

(Lipid-68 mer):
SEQ ID NO 2
5′-C18-ATTCGGTGAATAAGCACTCTCATAGTCCTCATCCAACTGCGCGTCTTGCATAGAG
CTGCTGACCCTAC-C12-3′

The synthesized oligonucleotides were recovered from the controlled pore glass (CPG) using ammonia (concentration of 28% or more). For this purpose, after synthesis, 1 ml of 28% ammonia water was added to the controlled pore glass (about 10 mg), which was then incubated at room temperature (about 25° C.) for about 30 minutes, after which the ammonia solution was recovered, thereby recovering the oligonucleotides as aqueous solution. The oligonucleotides treated under the above conditions were recovered such that DNA base protecting groups partially remained. These base protecting groups facilitate the dissolution of the oligonucleotides in an organic solvent and prevent the oligonucleotides from being degraded by enzymes or the like. These protecting groups may be removed using an organic solvent after recovery, so that the oligonucleotides can be analyzed by qPCR. The oligonucleotides in aqueous solution were quantified using UV absorbance at 260 nm.

(2) Preparation of Sample

An experiment wherein an aqueous solution of the oligonucleotide was dissolved in an organic solvent was carried out using a 15-ml conical tube (Corning). The oligonucleotide was dissolved in sterile water to reach an OD of about 50 per ml, but it was diluted to various concentrations in other experiments. As the phase transfer agent (PTA), a cationic phase transfer agent (+PTA) which can bond with the anionic region of the oligonucleotide by electrostatic attraction can be used. In this Examples, hexadecyltrimethylammonuim bromide (MW=364.5) was used as the phase transfer agent and it was dissolved in sterile water at a concentration of 1.3 nM, but it was dissolved at various concentrations in other experiments.

As the organic solvents, not only toluene and ether but also oils such as gasoline and diesel may be used. In this Example, gasoline (SK Energy Co., Ltd., Korea) and diesel (SK Energy Co., Ltd., Korea) were used as the vehicle oils.

(3) Experimental Procedure

An experiment wherein the aqueous oligonucleotide solution was dissolved in an organic solvent was carried out using a 15-ml conical tube (Corning).

The oligonucleotide was added to the conical tube to reach an OD of about 100, and then a cationic phase transfer agent (+PTA) was added to reach a sample volume of 2 ml, after which the same volume of an organic solvent was added. The conical tube containing the sample and the organic solvent was closed with a lid, and the sample was sufficiently mixed in a vortex for 1 minute or more. At this time, the polar portion of the oligonucleotide is neutralized by electrostatic bonding with the phase transfer agent so that it dissolves in the organic solvent. The mixed sample was centrifuged at 3,000 RPM for 10 into the aqueous layer and the organic solvent layer, and the lower aqueous layer was collected and measured for UV absorbance, whereby the amount of the oligonucleotide in the aqueous layer was determined. When the oligonucleotide remained in the aqueous layer, the organic solvent was recovered from the upper portion of the aqueous layer, the cationic phase transfer agent and the organic solvent were added thereto, and the mixing and separation processes were repeated.

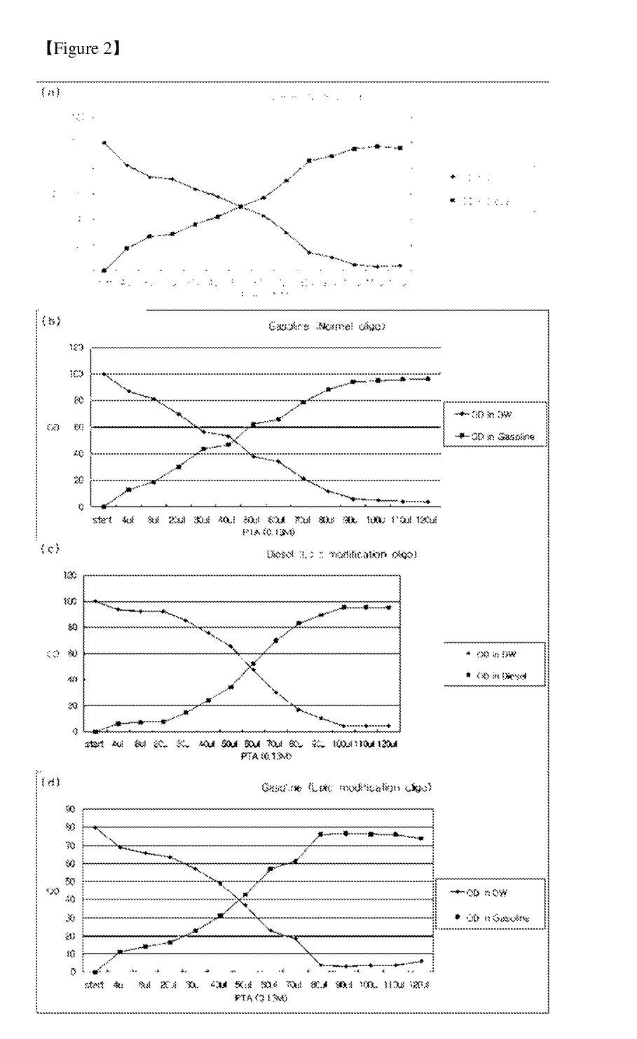

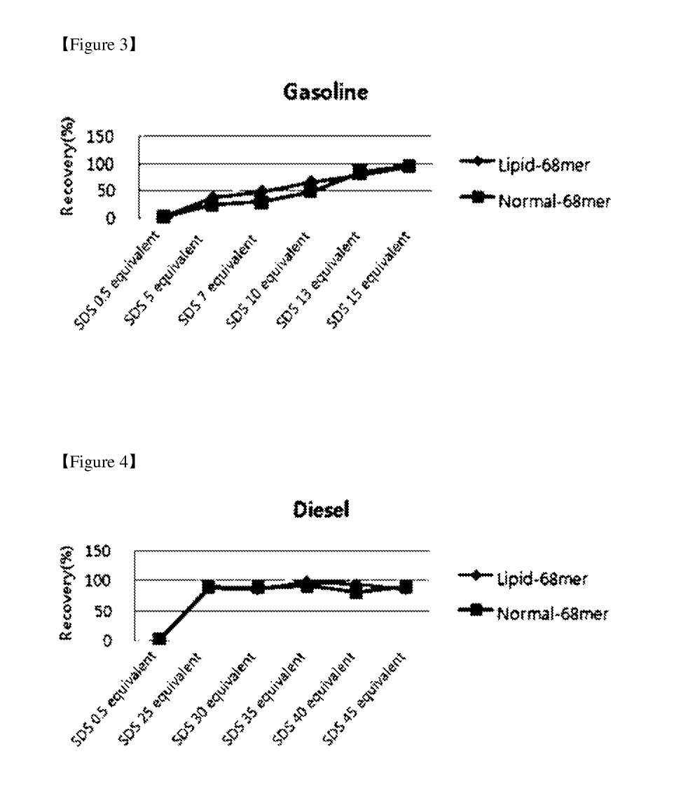

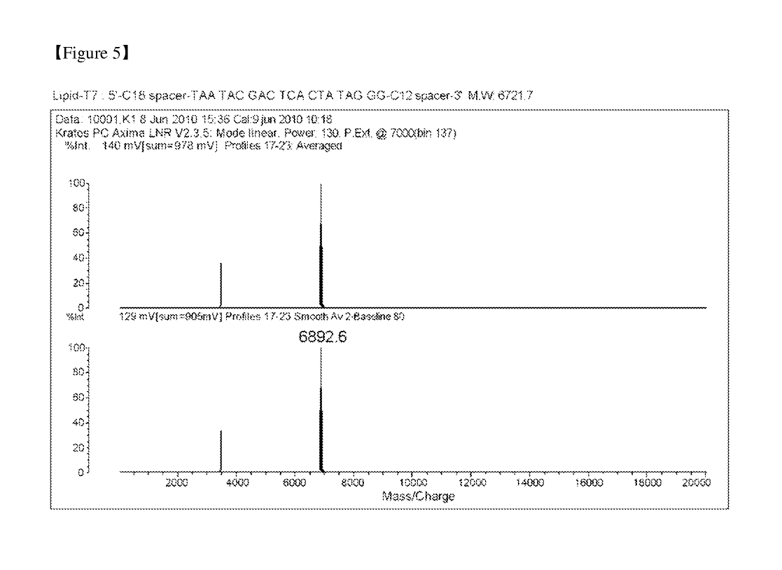

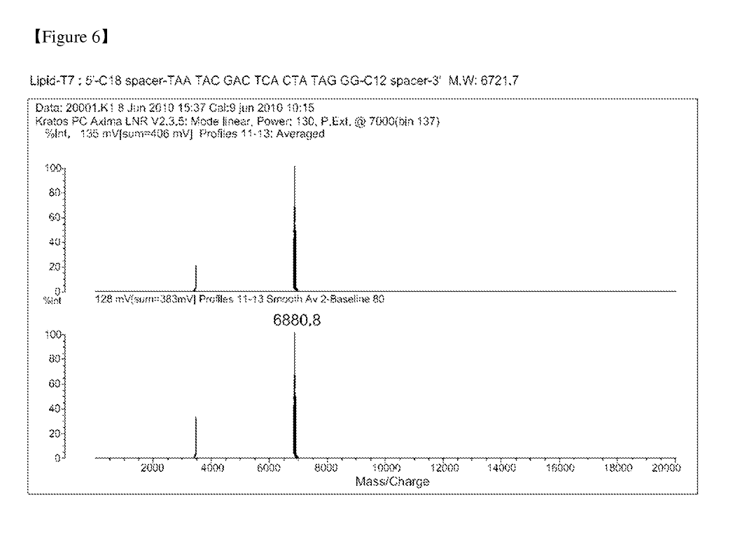

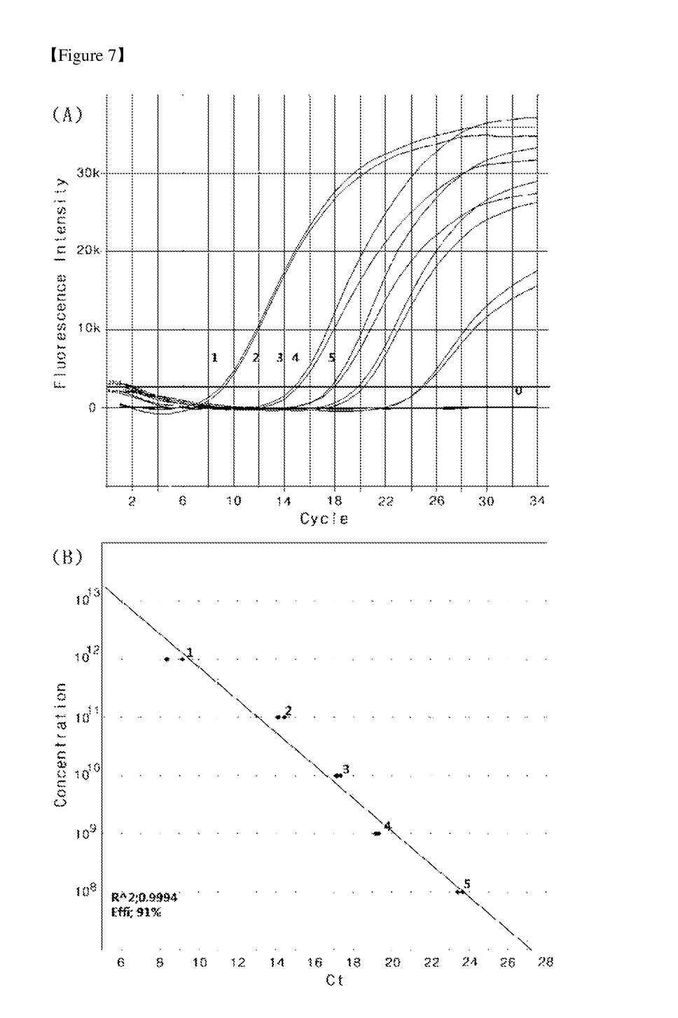

As can be seen from the results in FIG. 2, due to the use of the cationic phase transfer agent in an amount of 2 equivalents or more based on the oligonucleotide used, 90% or more of the oligonucleotide moved from the aqueous solution to the organic solvent, and the sample having a lipid added to both ends also showed similar results. In this Example, it could be seen that the partition coefficient (Pk) of the oligonucleotide in the organic solvent was about 20. Also, it could be seen that 95% or more of the oligonucleotide in the aqueous solution could be dissolved in the organic solvent by controlling the kind and ratio of the phase transfer agent and the organic solvent that were used.

### Example 2

### Recovery of Oligonucleotide Dissolved in Organic Solvent

Because the oligonucleotide dissolved in the organic solvent using the lipophilic properties of the cationic phase transfer agent (+PTA) and the base protecting groups has been stably dissolved in the organic solvent, it does not dissolve in any substantial amount in the water layer when the method of mixing it with water or boiling it with ammonia water is used.

Accordingly, if an anionic phase transfer agent (—PTA) capable of providing counter ions for the cationic phase transfer agent (+PTA) is added such that these counter ions are bonded with the cationic phase transfer agent (−PTA) in place of the oligonucleotide, the oligonucleotide can be extracted with water.

In this Example, each of the oligonucleotides (Lipid-68 mer and Normal-68 mer) dissolved in the organic solvent, obtained in Example 1, was diluted to reach an OD of about 30 per ml, but it was diluted at various concentrations in other experiments. The recovery of the oligonucleotide was expressed as a percentage relative to the initial amount added. As the anionic phase transfer agent (−PTA), SDS (sodium dodecyl sulfate; M.W:288.4) dissolved in sterile water at a concentration of 0.5 M was used, but any reagent may be used as the anionic phase transfer agent, as long as it can be dissolved in the organic solvent and can serve as counter ions for the cationic phase transfer agent (+PTA) bonded with the oligonucleotide in the organic solvent by electrostatic attraction.

In this Example, the same organic solvents as used in Example, containing the oligonucleotides dissolved therein, and the anionic phase transfer agent (−PTA), were used, and the aqueous layer containing the oligonucleotide dissolved therein was recovered.

TABLE 1
Recovery of oligonucleotide dissolved in gasoline

| | | SDS | | | | | | | Final |
|---|---|---|---|---|---|---|---|---|---|
| | Organic | Before | 0.5 | 5 | 7 | 10 | 13 | 15 | recovery |
| Sample | solvent | addition | equi. | equi. | equi. | equi. | equi. | equi. | rate |
| Lipid- | Gasoline | 100% | 2% | 39% | 50% | 67% | 80% | 94% | 94% |
| 68mer | | | | | | | | | |
| Normal- | | 100% | 4% | 24% | 29% | 50% | 84% | 96% | 96% |
| 68mer | | | | | | | | | |

TABLE 2
Recovery of oligonucleotide dissolved in diesel

| | | SDS | | | | | | | Final |
|---|---|---|---|---|---|---|---|---|---|
| | Organic | Before | 0.5 | 25 | 30 | 35 | 40 | 45 | recovery |
| Sample | solvent | addition | equi. | equi. | equi. | equi. | equi. | equi. | rate |
| Lipid- | Diesel | 100% | 2% | 88% | 85% | 99% | 93% | 87% | 99% |
| 68mer | | | | | | | | | |
| Normal- | | 100% | 3% | 89% | 88% | 91% | 82% | 90% | 91% |
| 68mer | | | | | | | | | |

As can be seen from the results in Tables 1 and 2 above, the amount of oligonucleotide recovered in the aqueous layer was determined by measuring the amount of SDS added and UV absorbance, and as a result, 90% or more of the sample could be recovered by adding about 15 equivalents of SDS for gasoline and about 35 equivalents of SDS for diesel.

TABLE 3

| | | Before | | | Final |
|---|---|---|---|---|---|
| | Organic | addition | SDS 15 | SDS 35 | recovery |
| Sample | solvent | of SDS | equi. | equi. | rate |
| Lipid- | diesel | 52% | — | 51% | 98% |
| 68mer | gasoline | 53% | 52% | — | 98% |
| Normal- | diesel | 28% | — | 2% | 97% |
| 68mer | gasoline | 31% | 30% | — | 98% |

As can be seen from the results in Table 3 above, the same results as shown in Tables 1 and 2 could be obtained even when the anionic phase transfer agent SDS was added at the same time at the final equivalent ratio.

### Example 3

### Analysis of Recovered Oligonucleotide

(1) Quantitative Analysis: MALDI-TOF Method

Whether the molecular structure of the oligonucleotide was modified during the process of dissolving the oligonucleotide in the solvent using the phase transfer agent and recovering the dissolved oligonucleotide was examined. As a sample, a short-length oligonucleotide (Lipid-22 mer) whose molecular weight can be determined by MALDI-TOF Mass was used.

SEQ ID NO 3: (Lipid-22 mer):
5′-C18-TAATACGACTCACTATAGGG-C12-3′ (MW = 6,722)

TABLE 4

| | | Before | | Final |
|---|---|---|---|---|
| | Organic | addition | SDS 0.5 | recovery |
| Sample | solvent | of SDS | equi. | rate |
| Lipid-22 mer | Gasoline | 100% | 4 | 94 |
| | Diesel | 100% | 10 | 96 |

The dissolution of the oligonucleotide in the organic solvent and the recovery of the dissolved oligonucleotide were performed in the same manner as in Examples 1 and 2. As can be seen from the results in Table 4 above, the oligonucleotide was recovered at a recovery rate of 90% or more while the molecular structure thereof was maintained without change during the treatment processes.

(2) Quantitative Analysis: qPCR (Quantitative Polymerase Chain Reaction) Method

In order to examine whether the oligonucleotide recovered after dissolution in the organic solvent can be analyzed by qPCR (quantitative polymerase chain reaction), the following experiment was carried out. The template having a length of 68mer, prepared in Examples 1 and 2, was used, and primers and a probe, specific for the template, were synthesized and used as follows.

Template Sequence:
5′-C18-ATTCGGTGAATAAGCACTCTCATAGTCCTCATCCAACTGCGC
GTCTTGCATAGAGCTGCTGACCCTAC-C12-3′
qPCR Primer/Probe Oligo:
Forward Primer sequence (SEQ ID NO 4):
5′-ATTCGGTGAATAAGCACTCTC-3′
Reverse Primer sequence (SEQ ID NO 5):
5′-GTAGGGTCAGCAGCTCTATG-3′
Probe sequence (SEQ ID NO 6):
5′-(FAM)-AGTCCTCATCCAACTGCGCGTCT-(Dabcyl)-3′

The probe used in this Example had a length of 23 mer and was labeled with the fluorescent dye FAM at the 5 end and with the fluorescent dye DABCYL at the 3 end. Real-time quantitative nucleic acid amplification for the oligonucleotide marker (template) of the present invention and samples 1, 2, 3, 4 and 5 was performed using AccuPower DualStar qPCR PreMix (Bioneer Co., Ltd) and AccuPower Greenstar qPCR PreMix (Bioneer Co. Ltd.). DNA quantification for the samples was performed using NANODROP2000/2000c (Thermo Scientific Co., Ltd.), and gasoline (SK Energy Co., Ltd.) was used as the oil to be labeled.

Samples for the qPCR analysis of oligonucleotide marker samples 1, 2, 3, 4 and 5 and a template were prepared.

(1) For the template, 1 ml of the template was diluted to 10¹³ copies/ml.

(2) For samples 1 to 5, 1 ml of the template was diluted to 10¹³-10⁹ copies/per ml.

(3) The template and samples 1 to 5 were cleaved and then purified by desalting.

(4) 1 ml of gasoline was prepared for each sample.

(5) 1 ml of distilled water containing each of the template and samples 1 to 5 dissolved therein, prepared in (3), was added to and dissolved in 1 ml of the prepared gasoline.

(6) 1 ml of each of the supernatants of the template and samples 1 to 5, separated in (5), was mixed with 1 ml of a Tamra cocktail containing SDS dissolved therein, and then each mixture was subjected to deprotection at 90 ? for 1 hour, after which 800 μl a of each supernatant was taken and dried.

(7) Each of the template and samples 1 to 5, prepared in (6), was added to and dissolved in 50 μl of distilled water.

(8) The template prepared in (7) was quantified by measuring absorbance at 260 nm using NANODROP2000/2000c (Thermo Scientific Inc.).

(9) Real-time nucleic acid amplification reaction (qPCR) experiments for the prepared samples were performed in the following manner.

The oligonucleotide marker template purified from gasoline was diluted to the copy numbers as shown in Table 5 below and was prepared in duplicate for each sample reaction.

TABLE 5

| sample | Condition (copies) | Vol. |
|---|---|---|
| 1, 1-1 | 1 × 10¹² | 5 μl |
| 2, 2-1 | 1 × 10¹¹ | |
| 3, 3-1 | 1 × 10¹⁰ | |
| 4, 4-1 | 1 × 10⁹ | |
| 5, 5-1 | 1 × 10⁸ | |
| 6, 6-1 | No template control | |

The quantified template purified from gasoline was prepared to the numbers of copies shown in Table 6 below and was prepared in duplicate for each sample reaction.

TABLE 6

| sample | Condition (copies) | Vol. |
|---|---|---|
| c1, 1-1 | 1 × 10¹¹ | 5 μl |
| c2, 2-1 | 1 × 10¹⁰ | |
| c3, 3-1 | 1 × 10⁹ | |
| c4, 4-1 | 1 × 10⁸ | |
| c5, 5-1 | 1 × 10⁷ | |
| c6, 6-1 | 1 × 10⁶ | |
| c7, 7-1 | No template control | |

A real-time nucleic acid amplification reaction had a final volume and was prepared as shown in Table 7 below.

TABLE 7
Reaction in one tube

| | |
|---|---|
| Sense primer (5 pmole/μl) | 12 p, 0.6 uM |
| Antisense primer (5 pmole/μl) | 12 p, 0.6 uM |
| Taqman probe (5 pmole/μl) | 15 p, 0.75 uM |
| Template (10⁹~10³ copies) | Each 5 μl |
| MgCl₂ (10 mM) | 0.4 μl |
| DEPC-distilled water | To a final volume |
| | of 20 μl |
| Total Volume | 20 μl |

For a real-time quantitative nucleic acid amplification reaction, AccuPower DualStar™qPCR PreMix (Bioneer Co., Ltd.) was prepared.

The reaction was performed using the real-time PCR machine Excycler™ (Bioneer Co., Ltd.) under the conditions shown in Table 8 below.

TABLE 8

| step | condition | Cycle |
|---|---|---|
| Pre-denaturation | 95° C., 5 min | 1 |
| Denaturation | 95° C., 5 sec | 35 |
| Annealing/Extension | 54° C., 9 sec | |
| Detection(Scan) | | |

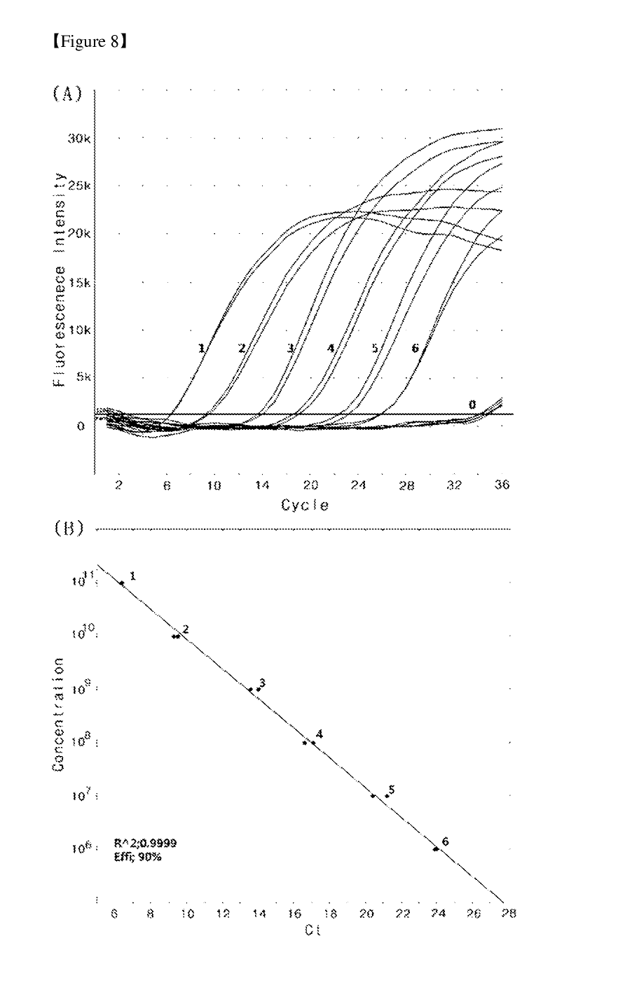

Oligonucleotide marker template samples 1, 2, 3, 4 and 5 which had been diluted to the respective numbers of copies were reacted with gasoline, after which they were recovered and purified. Then, a real-time quantitative amplification reaction was performed using each of purified template samples 1 to 5 with a probe and primers, and fluorescence graphs for the real-time quantitative amplification reaction are shown in FIG. 7A, in which the x-axis indicates the reaction recycle (hereinafter referred to as “Cy”), and the y-axis indicates the measured fluorescence according to the reaction cycle. Lanes 1 to 5 indicate the results of real-time quantitative amplification reactions when the numbers of copies were 1×10¹², 1×10¹¹, 1×10¹⁰, 1×10⁹ and 1×10⁸ copies, respectively. Lane 0 indicates the results of a reaction under NTC (no template control) conditions.

FIG. 7B is a graph showing the linearity of a quantification curve plotted using the fluorescence curves (for serial dilution conditions) shown in FIG. 7A, in which the y-axis indicates a log value for the measured fluorescence value, and the x-axis indicates the reaction cycle. Lanes 1 to 5 indicate the quantification curve for the real-time quantification nucleic acid amplification reactions for 1×10¹², 11×10¹¹, 1×10¹⁰, 1×10⁹ and 1×10⁸ copies, respectively. The quantification curve of FIG. 7B showed a PCR amplification efficiency of 91% and a PCR linearity (R² value) of 0.9994.

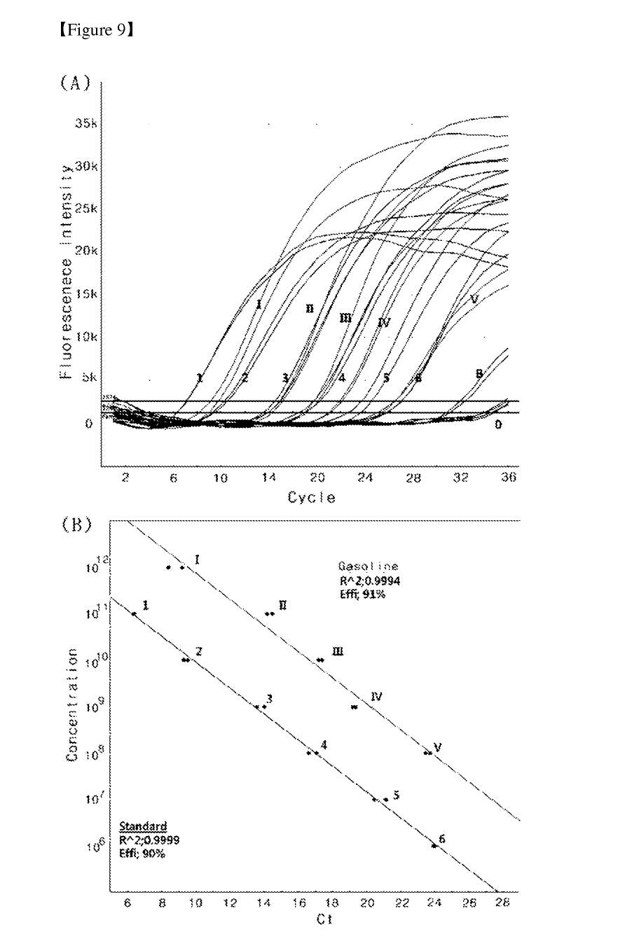

FIG. 8A is a set of fluorescence graphs showing real-time quantitative amplification reactions conducted using standard oligonucleotides (serially diluted to the respective copy numbers) as templates with a probe and primers. In FIG. 8A, the x-axis indicates the reaction recycle (hereinafter referred to as “Cy”), and the y-axis indicates the measured fluorescence value according to the reaction cycle. Lanes 1 to 6 indicate the results of real-time quantitative amplification reactions for copy numbers of 1×10¹¹, 1×10¹⁰, 1×10⁹, 1×10⁸, 1×10⁷ and 1×10⁶ copies per 20 μl reaction, respectively.

FIG. 8B is a graph showing the linearity of a quantification curve plotted using the fluorescence curves (for serial dilution conditions) shown in FIG. 8A, in which the y-axis indicates a log value for the measured fluorescence value, and the x-axis indicates the reaction cycle. Lanes 1 to 6 indicate the quantification curve for the real-time quantification nucleic acid amplification reactions for copy numbers of 1×10¹², 1×10¹¹, 1×10¹⁰, 1×10⁹ and 1×10⁸ copies per 20 μl reaction, respectively. The quantification curve of FIG. 8B showed a PCR amplification efficiency of 90% and a PCR linearity (R² value) of 0.9999.

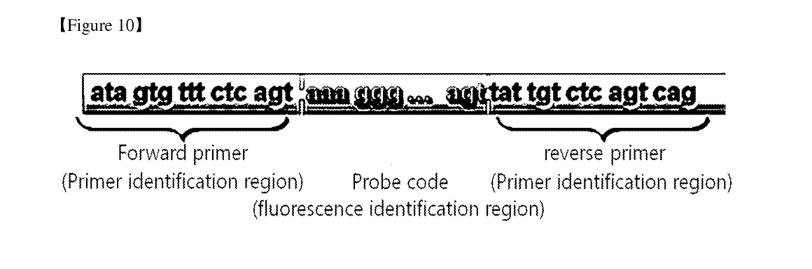

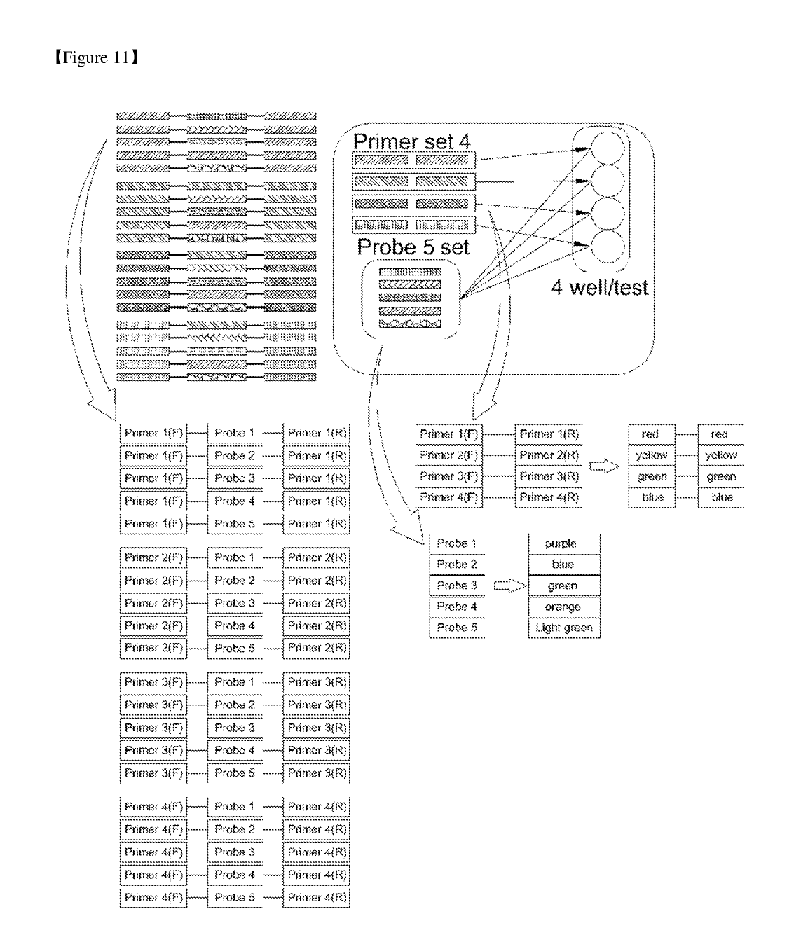

FIG. 9A is a set of overlapped fluorescence graphs showing the results of real-time quantitative nucleic acid amplification reactions for templates (blue) and samples 1(I), 2(II), 3(III), 4(IV) and 5(V, red. In FIG. 9A, the x-axis indicates the reaction recycle (“Cy”), and the y-axis indicates the measured fluorescence value according to the reaction cycle. Lanes 1 to 6 indicate the results of real-time quantitative amplification reactions for the templates for 1×10¹¹, 1×10¹⁰, 1×10⁹, 1×10⁸, 1×10⁷ and 1×10⁶ copies per 20 μl reaction, respectively, and Lanes I, II, III, IV and V indicate results of real-time quantitative amplification reactions for samples 1 to 5 (serially diluted to the respective copy numbers) for 1×10¹², 1×10¹¹, 1×10¹⁰, 1×10⁹ and 1×10⁸ copies per 20 μl a reaction, respectively. As can be seen in FIG. 9A, the efficiency of purification of the serially diluted samples was about 100 times lower than that of the templates. Also, the results for the serially diluted samples shown in the linearity of FIG. 9B were consistent with the results for the templates.

Real-time nucleic acid amplification reactions were performed using each of the oligonucleotide markers (diluted to the respective copy numbers) purified from gasoline and the standard oligonucleotide templates, and as a result, it could be seen that the oligonucleotide marker contained in gasoline was purified through the real-time nucleic acid amplification reaction using AccuPower DualStar qPCR PreMix, and the oligonucleotide markers (diluted to the respective copy numbers) purified from gasoline could be amplified to 1×10⁸ copies per 20 μl reaction.

From the above results, the oligonucleotide markers (templates) contained in gasoline and the oligonucleotide marker templates (diluted to the respective copy numbers) purified through the real-time nucleic acid amplification reactions using AccuPower DualStar qPCR PreMix could be amplified to a copy number of 1×10⁸ copies per 20 μl reaction.

### Example 4

### Labeling with a Combination of Oligonucleotide Markers

(1) Oligonucleotide Marker Identification Information

The gene sequences of a primer binding region (qPCR primer) that was used for the purpose of PCR amplification and to create a probe region for fluorescence measurement, linked to both ends of an oligonucleotide marker template, can be varied in various manners, so that they can be advantageously used as primers (forward and reverse primers) and probes which complementarily react with a specific oligonucleotide marker template.

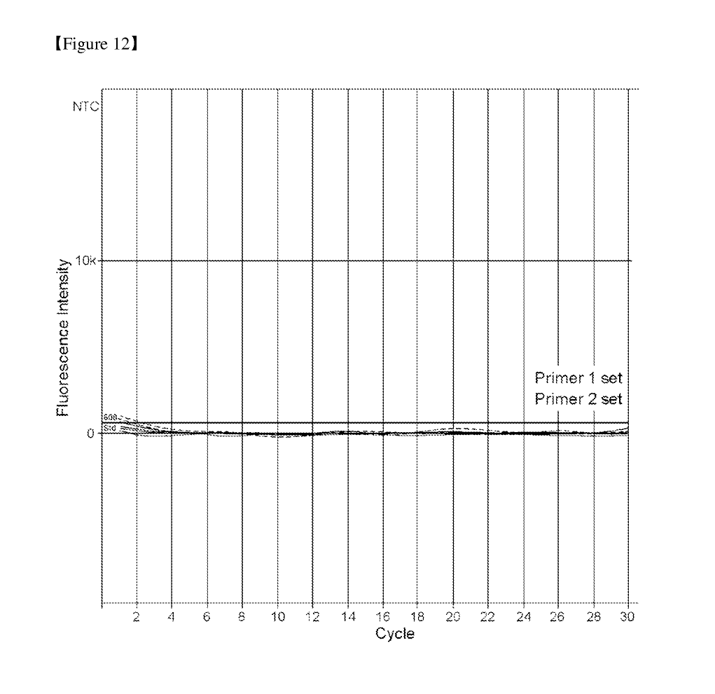

Specifically, if the sequences of a primer region and a probe region for fluorescence measurement, linked to both ends of an oligonucleotide marker template, are divided into 20 colors, a combination of four primer sets (red, yellow, green and blue) and five probes (purple, blue, green, orange and light green) can be exhibited as shown in FIG. 11. Among them, five is taken and combined with each other, about 1500 identification codes can then be produced, and more than several tens of thousands of various barcodes can be produced.

For example, as shown in FIG. 11, if two templates corresponding to full red and full green are labeled, red in the first well and green in the third well, from top to bottom of among four wells, can be exhibited. Alternatively, if purple and blue in the second well and green in the fourth well are exhibited, it can be seen that three oligonucleotide markers corresponding to a yellow primer/purple probe combination template, a yellow primer/blue probe template combination and a blue primer/green probe combination template are labeled.

A total of 20 templates, and 4 primer sets and five probes, which are specific for the templates, were constructed as follows:

Template Sequence: 5′->3′direction
#1-1 (SEQ ID NO 7):
C18 Spacer-ACAGGTAGGTAAGGTTCATGGTACCCGAACCAAGACGCATCTACCGGGGTCTGA
ATGACCAGAAGCACCT-C12 spacer
#1-2 (SEQ ID NO 8):
C18 Spacer-ACAGGTAGGTAAGGTTCATGGACGCTCCTAGTGCCGACTCCTACGTCCTACTGAA
TGACCAGAAGCACCT-C12 spacer
#1-3 (SEQ ID NO 9):
C18 Spacer-ACAGGTAGGTAAGGTTCATGGATTCGCCCTCGGATGCTGTCTCAGCGAGTCTGAA
TGACCAGAAGCACCT-C12 spacer
#1-4 (SEQ ID NO 10):
C18 Spacer-ACAGGTAGGTAAGGTTCATGGTCTGCCACCCGTGAGCGAATCGTCAGTCACTGA
ATGACCAGAAGCACCT-C12 spacer
#1-5 (SEQ ID NO 11):
C18 Spacer-ACAGGTAGGTAAGGTTCATGGAGGTTACCGAGACACCTGTGCATCCGCTCCTGA
ATGACCAGAAGCACCT-C12 spacer
#2-1 (SEQ ID NO 12):
C18 Spacer-GACCACGTCGTTCAGAATAAGTACCCGAACCAAGACGCATCTACCGGGGTGTAA
GCAGGTTATGTTGCCG-C12 spacer
#2-2 (SEQ ID NO 13):
C18 Spacer-GACCACGTCGTTCAGAATAAGACGCTCCTAGTGCCGACTCCTACGTCCTAGTAAG
CAGGTTATGTTGCCG-C12 spacer
#2-3 (SEQ ID NO 14):
C18 Spacer-GACCACGTCGTTCAGAATAAGATTCGCCCTCGGATGCTGTCTCAGCGAGTGTAAG
CAGGTTATGTTGCCG-C12 spacer
#2-4 (SEQ ID NO 15):
C18 Spacer-GACCACGTCGTTCAGAATAAGTCTGCCACCCGTGAGCGAATCGTCAGTCAGTAA
GCAGGTTATGTTGCCG-C12 spacer
#2-5 (SEQ ID NO 16):
C18 Spacer-GACCACGTCGTTCAGAATAAGAGGTTACCGAGACACCTGTGCATCCGCTCGTAA
GCAGGTTATGTTGCCG-C12 spacer
#3-1 (SEQ ID NO 17):
C18 Spacer-GACCGTTCTATTAAGGCAAGCTACCCGAACCAAGACGCATCTACCGGGGTCTCTG
CGATCTTCTGCTCTA-C12 spacer
#3-2 (SEQ ID NO 18):
C18 Spacer-GACCGTTCTATTAAGGCAAGCACGCTCCTAGTGCCGACTCCTACGTCCTACTCTG
CGATCTTCTGCTCTA-C12 spacer
#3-3 (SEQ ID NO 19):
C18 Spacer-GACCGTTCTATTAAGGCAAGCATTCGCCCTCGGATGCTGTCTCAGCGAGTCTCTG
CGATCTTCTGCTCTA-C12 spacer
#3-4 (SEQ ID NO 20):
C18 Spacer-GACCGTTCTATTAAGGCAAGCTCTGCCACCCGTGAGCGAATCGTCAGTCACTCTG
CGATCTTCTGCTCTA-C12 spacer
#3-5 (SEQ ID NO 21):
C18 Spacer-GACCGTTCTATTAAGGCAAGCAGGTTACCGAGACACCTGTGCATCCGCTCCTCTG
CGATCTTCTGCTCTA-C12 spacer
#4-1 (SEQ ID NO 22):
C18 Spacer-CGTGTCATGTTGTACCTAAGCTACCCGAACCAAGACGCATCTACCGGGGTCTTCA
AGTCGAGATACGCCT-C12 spacer
#4-2 (SEQ ID NO 23):
C18 Spacer-CGTGTCATGTTGTACCTAAGCACGCTCCTAGTGCCGACTCCTACGTCCTACTTCA
AGTCGAGATACGCCT-C12 spacer
#4-3 (SEQ ID NO 24):
C18 Spacer-CGTGTCATGTTGTACCTAAGCATTCGCCCTCGGATGCTGTCTCAGCGAGTCTTCA
AGTCGAGATACGCCT-C12 spacer
#4-4 (SEQ ID NO 25):
C18 Spacer-CGTGTCATGTTGTACCTAAGCTCTGCCACCCGTGAGCGAATCGTCAGTCACTTCA
AGTCGAGATACGCCT-C12 spacer
#4-5 (SEQ ID NO 26):
C18 Spacer-CGTGTCATGTTGTACCTAAGCAGGTTACCGAGACACCTGTGCATCCGCTCCTTCA
AGTCGAGATACGCCT-C12 spacer
Primer sequence: 4 sets
Forward primer #1 (SEQ ID NO 27):
5′-ACAGGTAGGTAAGGTTCATGG-3′
Reverse primer #1 (SEQ ID NO 28):
5′-AGGTGCTTCTGGTCATTCAG-3′
Forward primer #2 (SEQ ID NO 29):
5′-GACCACGTCGTTCAGAATAAG-3′
Reverse primer #2 (SEQ ID NO 30):
5′-CGGCAACATAACCTGCTTAC-3′
Forward primer #3 (SEQ ID NO 31):
5′-GACCGTTCTATTAAGGCAAGC-3′
Reverse primer #3 (SEQ ID NO 32):
5′-TAGAGCAGAAGATCGCAGAG-3′
Forward primer #4 (SEQ ID NO 33):
5′-CGTGTCATGTTGTACCTAAGC-3′
Reverse primer #4 (SEQ ID NO 34):
5′-AGGCGTATCTCGACTTGAAG-3′
Probe sequences: five
probe #1 (SEQ ID NO 35):
5′-(FAM)-ACCCGAACCAAGACGCATCTACCG-(BHQ1)-3′
probe #2 (SEQ ID NO 36):
5′-(TET)-CGCTCCTAGTGCCGACTCCTACG-(BHQ1)-3′
probe #3 (SEQ ID NO 37):
5′-(Tamra)-TTCGCCCTCGGATGCTGTCTCA-(BHQ1)-3′
probe #4 (SEQ ID NO 38):
5′-(Texas Red)-TGCCACCCGTGAGCGAATCGT-(BHQ2)-3′
probe #5 (SEQ ID NO 39):
5′-(Cy5)-ACCGAGACACCTGTGCATCCGC-(BHQ2)-3′

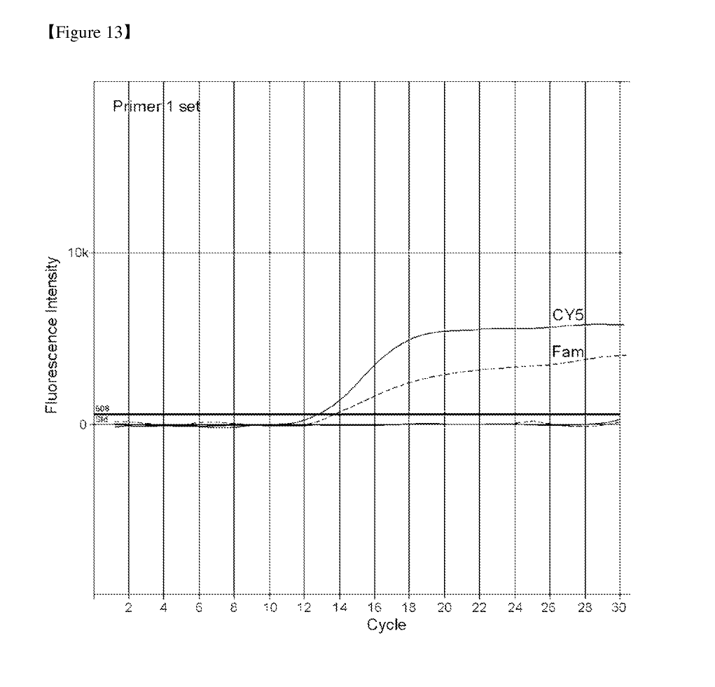

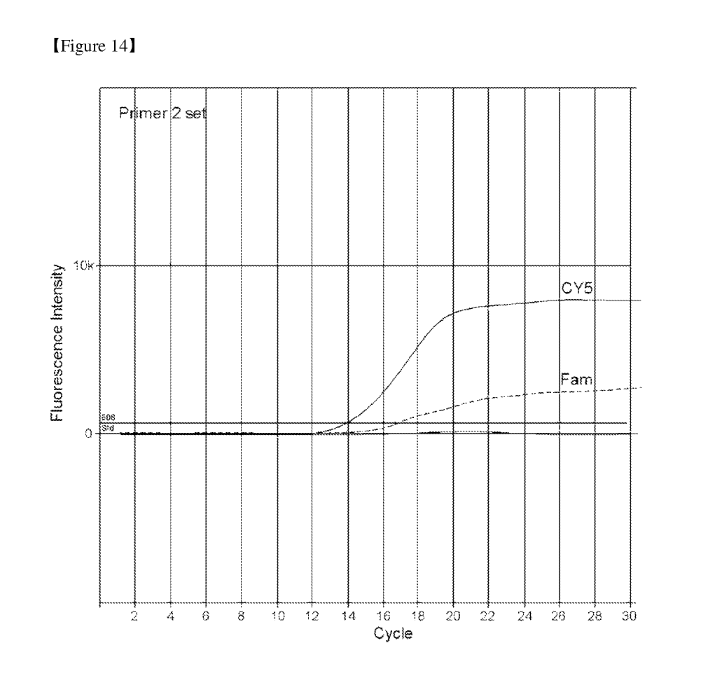

Each of primer set #1 (SEQ ID NO: 27/SEQ ID NO: 28) and primer set #2 (SEQ ID NO: 29/SEQ ID NO: 30) was introduced into qPCR reaction tubes containing probe #1 (SEQ ID NO 35) (FAM, green) and probe #5 (SEQ ID NO 39) (Cy5, red). The 20 templates were added to each of the primer sets to prepare four template mixture samples, after which qPCR reactions were performed using the template samples. As a result, as can be seen in FIGS. 12 to 14, a PCR reaction did not occur under the NTC (no template control) conditions. On the other hand, in the reaction containing each of primer sets #1 and #2, multiple reactivity could be observed in the CY5 probe and the FAM probe.

[Sequence List Text]

SEQ ID NO: 1 is the nucleotide sequence of an oligonucleotide of Normal-68 mer according to the present invention.

SEQ ID NO: 2 is the nucleotide sequence of an oligonucleotide of lipid-68 mer according to the present invention.

SEQ ID NO: 3 is the nucleotide sequence of an oligonucleotide of Lipid-22 mer according to the present invention.

SEQ ID NO: 4 is the nucleotide sequence of a forward qPCR primer for analyzing a recovered oligonucleotide according to the present invention.

SEQ ID NO: 5 is the nucleotide sequence of a reverse qPCR primer for analyzing a recovered oligonucleotide according to the present invention.

SEQ ID NO: 6 is the nucleotide sequence of a probe for analyzing a recovered oligonucleotide according to the present invention.

SEQ ID NO: 7 is the nucleotide sequence of a specific oligonucleotide marker template (#1-1) according to the present invention.

SEQ ID NO: 8 is the nucleotide sequence of a specific oligonucleotide marker template (#1-2) according to the present invention.

SEQ ID NO: 9 is the nucleotide sequence of a specific oligonucleotide marker template (#1-3) according to the present invention.

SEQ ID NO: 10 is the nucleotide sequence of a specific oligonucleotide marker template (#1-4) according to the present invention.

SEQ ID NO: 11 is the nucleotide sequence of a specific oligonucleotide marker template (#1-5) according to the present invention.

SEQ ID NO: 12 is the nucleotide sequence of a specific oligonucleotide marker template (#2-1) according to the present invention.

SEQ ID NO: 13 is the nucleotide sequence of a specific oligonucleotide marker template (#2-2) according to the present invention.

SEQ ID NO: 14 is the nucleotide sequence of a specific oligonucleotide marker template (#2-3) according to the present invention.

SEQ ID NO: 15 is the nucleotide sequence of a specific oligonucleotide marker template (#2-4) according to the present invention.

SEQ ID NO: 16 is the nucleotide sequence of a specific oligonucleotide marker template (#2-5) according to the present invention.

SEQ ID NO: 17 is the nucleotide sequence of a specific oligonucleotide marker template (#3-1) according to the present invention.

SEQ ID NO: 18 is the nucleotide sequence of a specific oligonucleotide marker template (#3-2) according to the present invention.

SEQ ID NO: 19 is the nucleotide sequence of a specific oligonucleotide marker template (#3-3) according to the present invention.

SEQ ID NO: 20 is the nucleotide sequence of a specific oligonucleotide marker template (#3-4) according to the present invention.

SEQ ID NO: 21 is the nucleotide sequence of a specific oligonucleotide marker template (#3-5) according to the present invention.

SEQ ID NO: 22 is the nucleotide sequence of a specific oligonucleotide marker template (#4-1) according to the present invention.

SEQ ID NO: 23 is the nucleotide sequence of a specific oligonucleotide marker template (#4-2) according to the present invention.

SEQ ID NO: 24 is the nucleotide sequence of a specific oligonucleotide marker template (#4-3) according to the present invention.

SEQ ID NO: 25 is the nucleotide sequence of a specific oligonucleotide marker template (#4-4) according to the present invention.

SEQ ID NO: 26 is the nucleotide sequence of a specific oligonucleotide marker template (#4-5) according to the present invention.

SEQ ID NO: 27 is the nucleotide sequence of a forward PCR primer (#1) specific for 20 templates according to the present invention.

SEQ ID NO: 28 is the nucleotide sequence of a reverse PCR primer (#1) specific for 20 templates according to the present invention.

SEQ ID NO: 29 is the nucleotide sequence of a forward PCR primer (#2) specific for 20 templates according to the present invention.

SEQ ID NO: 30 is the nucleotide sequence of a reverse PCR primer (#2) specific for 20 templates according to the present invention.

SEQ ID NO: 31 is the nucleotide sequence of a forward PCR primer (#3) specific for 20 templates according to the present invention.

SEQ ID NO: 32 is the nucleotide sequence of a reverse PCR primer (#3) specific for 20 templates according to the present invention.

SEQ ID NO: 33 is the nucleotide sequence of a forward PCR primer (#4) specific for 20 templates according to the present invention.

SEQ ID NO: 34 is the nucleotide sequence of a reverse PCR primer (#4) specific for 20 templates according to the present invention.

SEQ ID NO: 35 is the nucleotide sequence of a probe (#1) specific for 20 templates according to the present invention.

SEQ ID NO: 36 is the nucleotide sequence of a probe (#2) specific for 20 templates according to the present invention.

SEQ ID NO: 37 is the nucleotide sequence of a probe (#3) specific for 20 templates according to the present invention.

SEQ ID NO: 38 is the nucleotide sequence of a probe (#4) specific for 20 templates according to the present invention.

SEQ ID NO: 38 is the nucleotide sequence of a probe (#5) specific for 20 templates according to the present invention.

1. An oligonucleotide marker for identifying a material, the oligonucleotide marker being an oligonucleotide linked with a cationic phase transfer agent and comprising a probe sequence for real-time polymerase chain reaction (PCR) and primer sequences linked to both ends of the probe sequence.

2. The oligonucleotide marker of claim 1, wherein the oligonucleotide is blocked at its end by a blocking agent.

3. The oligonucleotide marker of claim 2, wherein the blocking agent is free of a chemical substance or an end group, which is a lipid or a phosphate.

4. The oligonucleotide marker of claim 1, wherein the oligonucleotide comprises reactive nitrogen and oxygen moieties linked to an organic compound containing 1-50 carbon atoms.

5. The oligonucleotide marker of claim 1, wherein the oligonucleotide has a length of 20-1000 nucleotides.

6. A method for identifying a material, the method comprising adding the oligonucleotide marker of claim 1 to the material.

7. The method of claim 6, wherein the material is selected from the group consisting of paints for vehicle coating, lacquers, paints for traffic lines, petroleum, paint diluents, thinners, explosives, naturally occurring oils, paints for construction, organic solvents, adhesives, dyes, meats and fish.

8. A method for identifying a material, the method comprising:
1) extracting an oligonucleotide from a material labeled with the oligonucleotide marker of claim 1;
2) amplifying the extracted oligonucleotide by real-time PCR using primers and a probe; and
3) identifying the material, labeled with the oligonucleotide marker, using the real-time PCR product.

9. The method of claim 8, wherein the probe is a fluorescence-labeled probe.

10. The method of claim 8, wherein the extracting of 1) is performed using an anionic phase transfer agent.

11. The method of claim 8, wherein the material is a fat-soluble or water-soluble material.

12. The method of claim 11, wherein the material is selected from the group consisting of paints for vehicle coating, lacquers, paint for traffic lines, petroleum, paint diluents, thinners, explosives, naturally occurring oils, paints for construction, organic solvents, adhesives, dyes, meats and fishes.

13. The method of claim 8, wherein the identifying is performed by identifying the material by a combination of the oligonucleotide markers having different sequences, which are contained in the material.

14. A composition for identifying a material, the composition containing the oligonucleotide marker of claim 1.

15. The composition of claim 14, further comprising primers and a probe, which correspond to the oligonucleotide marker.


SEQUENCE LISTING (39 sequences)

SEQ ID NO: 1 - DNA, 68 bp, Artificial
  Synthetic construct
attcggtgaataagcactctcatagtcctcatccaactgcgcgtcttgcatagagctgctgaccctac

SEQ ID NO: 2 - DNA, 68 bp, Artificial; identical to SEQ ID NO: 1
  Synthetic construct

SEQ ID NO: 3 - DNA, 20 bp, Artificial
  Artifical construct
taatacgactcactataggg

SEQ ID NO: 4 - DNA, 21 bp, Artificial
  forward primer
attcggtgaataagcactctc

SEQ ID NO: 5 - DNA, 20 bp, Artificial
  reverse primer
gtagggtcagcagctctatg

SEQ ID NO: 6 - DNA, 23 bp, Artificial
  Probe
agtcctcatccaactgcgcgtct

SEQ ID NO: 7 - DNA, 70 bp, Artificial
  Synthetic construct
acaggtaggtaaggttcatggtacccgaaccaagacgcatctaccggggtctgaatgaccagaagcacct

SEQ ID NO: 8 - DNA, 70 bp, Artificial
  Synthetic construct
acaggtaggtaaggttcatggacgctcctagtgccgactcctacgtcctactgaatgaccagaagcacct

SEQ ID NO: 9 - DNA, 70 bp, Artificial
  Synthetic construct
acaggtaggtaaggttcatggattcgccctcggatgctgtctcagcgagtctgaatgaccagaagcacct

SEQ ID NO: 10 - DNA, 70 bp, Artificial
  Synthetic construct
acaggtaggtaaggttcatggtctgccacccgtgagcgaatcgtcagtcactgaatgaccagaagcacct

SEQ ID NO: 11 - DNA, 70 bp, Artificial
  Synthetic construct
acaggtaggtaaggttcatggaggttaccgagacacctgtgcatccgctcctgaatgaccagaagcacct

SEQ ID NO: 12 - DNA, 70 bp, Artificial
  Synthetic construct
gaccacgtcgttcagaataagtacccgaaccaagacgcatctaccggggtgtaagcaggttatgttgccg

SEQ ID NO: 13 - DNA, 70 bp, Artificial
  Synthetic construct
gaccacgtcgttcagaataagacgctcctagtgccgactcctacgtcctagtaagcaggttatgttgccg

SEQ ID NO: 14 - DNA, 70 bp, Artificial
  Synthetic construct
gaccacgtcgttcagaataagattcgccctcggatgctgtctcagcgagtgtaagcaggttatgttgccg

SEQ ID NO: 15 - DNA, 70 bp, Artificial
  Synthetic construct
gaccacgtcgttcagaataagtctgccacccgtgagcgaatcgtcagtcagtaagcaggttatgttgccg

SEQ ID NO: 16 - DNA, 70 bp, Artificial
  Synthetic construct
gaccacgtcgttcagaataagaggttaccgagacacctgtgcatccgctcgtaagcaggttatgttgccg

SEQ ID NO: 17 - DNA, 70 bp, Artificial
  Synthetic construct
gaccgttctattaaggcaagctacccgaaccaagacgcatctaccggggtctctgcgatcttctgctcta

SEQ ID NO: 18 - DNA, 70 bp, Artificial
  Synthetic construct
gaccgttctattaaggcaagcacgctcctagtgccgactcctacgtcctactctgcgatcttctgctcta

SEQ ID NO: 19 - DNA, 70 bp, Artificial
  Synthetic construct
gaccgttctattaaggcaagcattcgccctcggatgctgtctcagcgagtctctgcgatcttctgctcta

SEQ ID NO: 20 - DNA, 70 bp, Artificial
  Synthetic construct
gaccgttctattaaggcaagctctgccacccgtgagcgaatcgtcagtcactctgcgatcttctgctcta

SEQ ID NO: 21 - DNA, 70 bp, Artificial
  Synthetic construct
gaccgttctattaaggcaagcaggttaccgagacacctgtgcatccgctcctctgcgatcttctgctcta

SEQ ID NO: 22 - DNA, 70 bp, Artificial
  Synthetic construct
cgtgtcatgttgtacctaagctacccgaaccaagacgcatctaccggggtcttcaagtcgagatacgcct

SEQ ID NO: 23 - DNA, 70 bp, Artificial
  Synthetic construct
cgtgtcatgttgtacctaagcacgctcctagtgccgactcctacgtcctacttcaagtcgagatacgcct

SEQ ID NO: 24 - DNA, 70 bp, Artificial
  Synthetic construct
cgtgtcatgttgtacctaagcattcgccctcggatgctgtctcagcgagtcttcaagtcgagatacgcct

SEQ ID NO: 25 - DNA, 70 bp, Artificial
  Synthetic construct
cgtgtcatgttgtacctaagctctgccacccgtgagcgaatcgtcagtcacttcaagtcgagatacgcct

SEQ ID NO: 26 - DNA, 70 bp, Artificial
  Synthetic construct
cgtgtcatgttgtacctaagcaggttaccgagacacctgtgcatccgctccttcaagtcgagatacgcct

SEQ ID NO: 27 - DNA, 21 bp, Artificial
  forward primer 1
acaggtaggtaaggttcatgg

SEQ ID NO: 28 - DNA, 20 bp, Artificial
  reverse primer 1
aggtgcttctggtcattcag

SEQ ID NO: 29 - DNA, 21 bp, Artificial
  forward primer 2
gaccacgtcgttcagaataag

SEQ ID NO: 30 - DNA, 20 bp, Artificial
  reverse primer 2
cggcaacataacctgcttac

SEQ ID NO: 31 - DNA, 21 bp, Artificial
  forward primer 3
gaccgttctattaaggcaagc

SEQ ID NO: 32 - DNA, 20 bp, Artificial
  reverse primer 3
tagagcagaagatcgcagag

SEQ ID NO: 33 - DNA, 21 bp, Artificial
  forward primer 4
cgtgtcatgttgtacctaagc

SEQ ID NO: 34 - DNA, 20 bp, Artificial
  reverse primer 4
aggcgtatctcgacttgaag

SEQ ID NO: 35 - DNA, 24 bp, Artificial
  probe
acccgaaccaagacgcatctaccg

SEQ ID NO: 36 - DNA, 23 bp, Artificial
  probe
cgctcctagtgccgactcctacg

SEQ ID NO: 37 - DNA, 22 bp, Artificial
  probe
ttcgccctcggatgctgtctca

SEQ ID NO: 38 - DNA, 21 bp, Artificial
  probe
tgccacccgtgagcgaatcgt

SEQ ID NO: 39 - DNA, 22 bp, Artificial
  probe
accgagacacctgtgcatccgc